(19) United States
(12) Patent Application Publication
(10) Pub. No.: US 2026/0284355 A1
(43) Pub. Date: Sep. 24, 2026

(54) Catheter Tip Structure and Method of Manufacture

(71) Applicant: Bard Access Systems, Inc., Salt Lake City, UT (US)

(72) Inventors: Juan Sepulveda, Centerville, UT (US); Glade H. Howell, Draper, UT (US)

(21) Appl. No.: 19/694,751

(22) Filed: Jun. 1, 2026

Related U.S. Application Data
(63) Continuation of application No. 17/969,626, filed on Oct. 19, 2022, now Pat. No. 12,642,939.
(60) Provisional application No. 63/270,496, filed on Oct. 21, 2021.

(51) Int. Cl. A61M 25/00; B29C 33/48

(52) U.S. Cl. CPC A61M 25/0068; A61M 25/0009; A61M 25/001; A61M 25/003; A61M 25/0043; B29C 33/485; A61M 2025/0018; A61M 25/0026; A61M 2025/0031

(57) ABSTRACT

A method of forming a catheter includes extruding a catheter body from a first material and forming a distal tip onto the catheter body. The catheter body can include a first lumen, a second lumen, and a third lumen. Forming the distal tip onto the catheter body can include positioning a proximal end of a mandrel in a distal end of the third lumen, positioning a portion of the mandrel, a distal end of the catheter body, and a pellet of a second material in a die, and deforming the pellet around the mandrel to form the distal tip. The second material can be different from the first material. The method can further include removing the mandrel from the catheter body and the distal tip to provide a passage from the third lumen at the distal end of the catheter body through the distal tip.

### PRIORITY

This application is a continuation of U.S. Patent Application No. 17/969,626, filed October 19, 2022, now U.S. Patent No. 12,642,939, which claims the benefit of priority to U.S. Provisional Application No. 63/270,496, filed October 21, 2021, each of which is incorporated by reference in its entirety into this application.

### SUMMARY

Briefly summarized, embodiments disclosed herein are directed to distal tip structures for a catheter, such as a rapidly insertable central catheter (RICC), and associated methods of manufacture. RICC systems include a catheter having a multi-luminal section defining two or more lumen, an access section defining a single lumen, and a dilator section disposed therebetween. The configuration of the RICC catheter allows a clinician to access the vasculature, dilate the access site, and place the multi-luminal section in a single step, mitigating the introduction and removal of multiple tools to achieve each of these steps separately. Forming the RICC catheter requires coupling the three different structures of the multi-luminal section, dilator section, and access section together, while maintaining a smooth outer profile. Each of the three different structures are required to display different mechanical properties to fulfil the respective roles in the placement process. Disclosed herein is a method of manufacture of a distal tip structure of a RICC catheter.

Disclosed herein is a method of forming a catheter including, extruding a multi-luminal section of a catheter body having a first catheter lumen and a second catheter lumen, forming a distal tip structure of the catheter body including, placing a first tine of a bifurcated plug within the first catheter lumen, placing a second tine of the bifurcated plug within the second catheter lumen, a distal tip of the bifurcated plug extending distally of a distal end of the catheter body, and forming the bifurcated plug into the distal tip structure, coupled to the multi-luminal section.

In some embodiments, the multi-luminal section is formed of a first material and the bifurcated plug is formed of a second material, different from the first material. In some embodiments, the first material includes a more compliant, or softer durometer, mechanical properties relative to the second material. In some embodiments, the distal tip structure includes one or both of a dilator section and an access section. In some embodiments, the step of forming the bifurcated plug into the distal tip structure further includes placing a portion of the bifurcated plug and a distal end of the multi-luminal section into a die and applying one or more of pressure, thermal energy, radio frequency energy, or ultrasonic energy to plastically deform the bifurcated plug into the distal tip structure. In some embodiments the method further includes placing a mandrel within a third lumen of the multi-luminal section to define a portion of a distal lumen within the distal tip structure. In some embodiments, the bifurcated plug includes one of a rod or a tube folded in half and wherein a first end defines the first tine, and the second end defines the second tine.

Also disclosed is a method of forming a distal tip structure for a catheter including, forming a catheter body including a multi-luminal section having a first material and defining a first lumen and a second lumen, placing a proximal end of a mandrel within a distal end of the first lumen, placing a portion of the mandrel, a distal end of the multi-luminal section, and a pellet of a second material, different from the first material, within a die, and plastically deforming the pellet around the portion of the mandrel to form the distal tip structure coupled to the distal end of the multi-luminal section.

In some embodiments, the first material is relatively more compliant, elastically deformable, or a softer durometer relative to the second material. In some embodiments, the distal tip structure includes a dilator section and a portion of an access section. In some embodiments, the distal tip structure includes a dilator section having a recess configured to receive a proximal portion of an access section therein. In some embodiments, the method further includes coupling the proximal portion of the access section with the recess using adhesive, bonding, or welding.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a RICC system, in accordance with embodiments disclosed herein.

FIG. 1B shows a side view of a catheter of the RICC system of FIG. 1A, in accordance with embodiments disclosed herein.

FIG. 2 shows close up detail of a distal portion of the catheter of FIG. 1B, in accordance with embodiments disclosed herein.

FIGS. 3-6 show various cross-sectional views of the distal portion of FIG. 2, in accordance with embodiments disclosed herein.

FIG. 7 shows an exemplary method of forming a distal tip structure of a catheter body, in accordance with embodiments disclosed herein.

FIGS. 8A-8C show an exemplary method of forming a distal tip structure of a catheter body, in accordance with embodiments disclosed herein.

FIGS. 9A-9C show an exemplary method of forming a distal tip structure of a catheter body, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

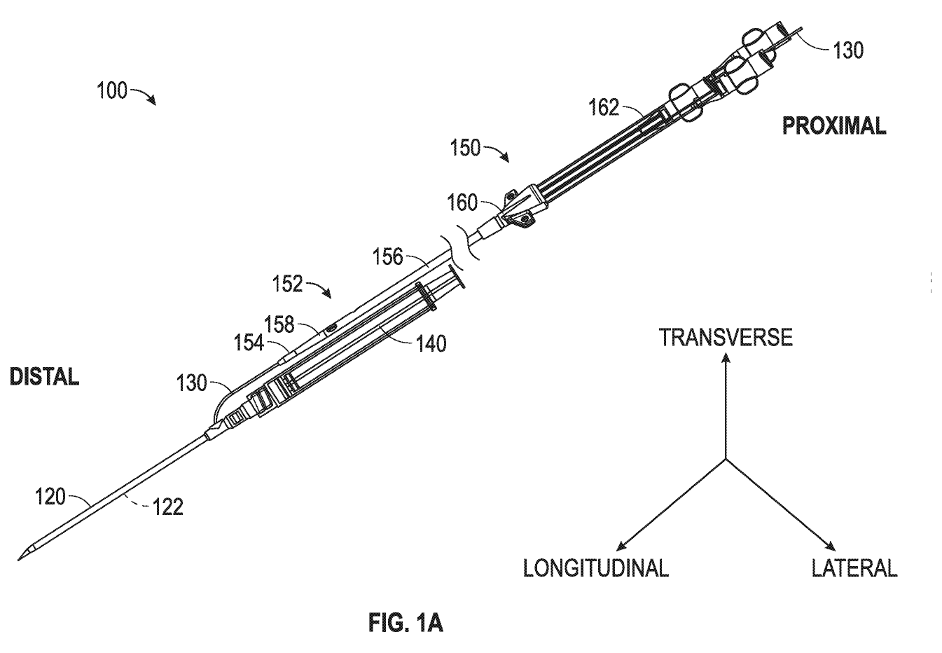

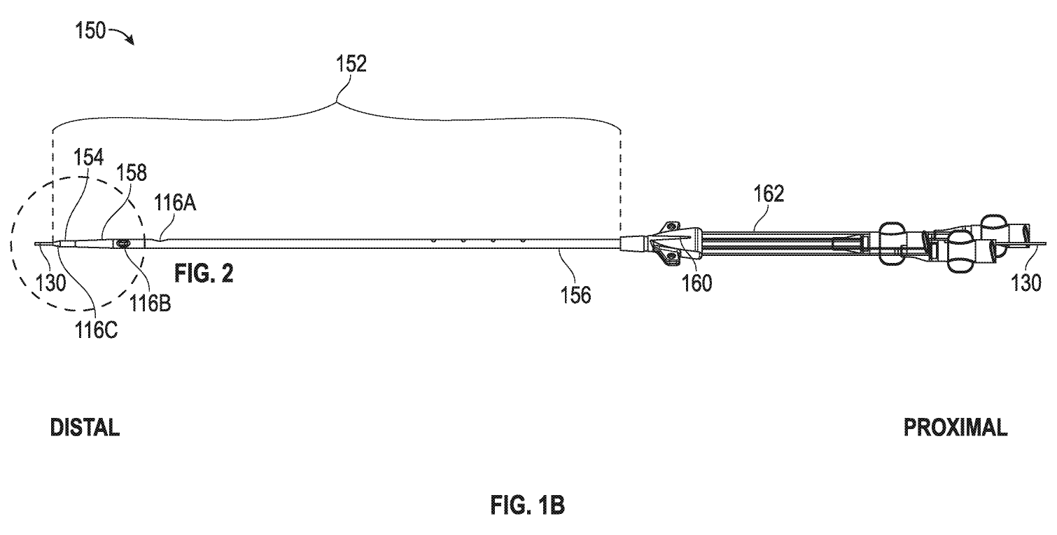

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIGS. 1A-1B show details of an exemplary Rapidly Insertable Central Catheter (RICC) placement system (“placement system”) 100 generally including a needle 120, a guidewire 130, a syringe system 140, and a RICC catheter 150. The RICC catheter 150 can generally include a catheter body 152 supported at a proximal end by a catheter hub (“hub”) 160. The hub 160 can include one or more extension legs 162 extending proximally therefrom. Each extension leg of the one or more extension legs 162 can be in fluid communication with a lumen of the catheter body 152. The catheter body 152 can include an access section 154, a multi-luminal section 156, and a dilator section 158 disposed therebetween. A guidewire 130 can extend through a lumen of the RICC catheter 150 from a proximal end of an extension leg 162, to a distal tip of the access section 154.

In an exemplary method of placing the RICC catheter 150, the needle 120 can be urged distally into the patient and access a vasculature, forming an insertion site. A syringe system 140, or similar device can draw a fluid flow proximally through a needle lumen 122 to observe a color and/or pulsatile flow and confirm correct vascular access. Once correct vascular access has been confirmed, the guidewire 130 can then be advanced through the needle lumen 122 and into the vasculature to maintain patency of the insertion site. The needle 120 and syringe system 140 assembly can then be withdrawn proximally. In an embodiment, a distal tip of the guidewire 130 can reside within the needle lumen 122 during venipuncture, which can expedite accessing the vasculature once venous access is confirmed and can maintain patency of the insertion site.

The RICC 150 can then be advanced over the guidewire 130 and into the vasculature. The access section 154 of the RICC 150, having only a single lumen, defines a relatively small outer diameter and can enter the vasculature over the guidewire 130, anchoring the insertion site. The dilator section 158 can then dilate the insertion site to allow the relatively larger diameter, multi-luminal section 156 which defines two or more lumen, to enter the vasculature. Once the RICC 150 has been placed, the guidewire 130 can be withdrawn proximally. Further details and embodiments of RICC systems 100 can be found, for example, in U.S. 10,376,675, U.S. 2019/0255294, U.S. 2021/0069471, U.S. 2021/0085927, U.S. 2021/0113809, U.S. 2021/0113810, U.S. 2021/0121661, U.S. 2021/0228843, U.S. 2021/0283368, U.S. 2021/0283381, U.S. 2021/0322729, U.S. 2021/0330941, U.S. 2021/0330942, U.S. 2021/0361915, U.S. 2021/0379336, U.S. 2021/0402142, U.S. 2021/0402149, U.S. 2021/0402153, U.S. 2021/0121667, U.S. 2022/0001138, U.S. 2022/0032013, U.S. 2022/0032014, U.S. 2022/0062528, U.S. 2022/0126064, U.S. 2022/0152368, U.S. 2022/0176081, U.S. 2022/0176082, U.S. 2022/0193376, U.S. 2022/0193377, U.S. 2022/0193378, U.S. 2022/0193379, and U.S. 2022/0296862, each of which is incorporated by reference in its entirety into this application.

As set forth herein, different portions of the RICC catheter 150 are required to perform different functions and as such are required to display different mechanical properties. For example, the access section 154 and/or the dilator section 158 can include more rigid mechanical properties, or a harder durometer material, relative to the multi-luminal section 156. As such, the access section 154 and dilator section 158 can withstand greater axial forces without kinking or collapsing, as theses sections are urged distally forming and dilating the insertion site. The multi-luminal section 156 can be formed of a relatively softer durometer, or a more compliant material, to facilitate negotiating through tortuous vascular pathways. Forming the RICC catheter 150 requires the coupling together of these different structures, formed of different materials, while maintaining a smooth abluminal surface.

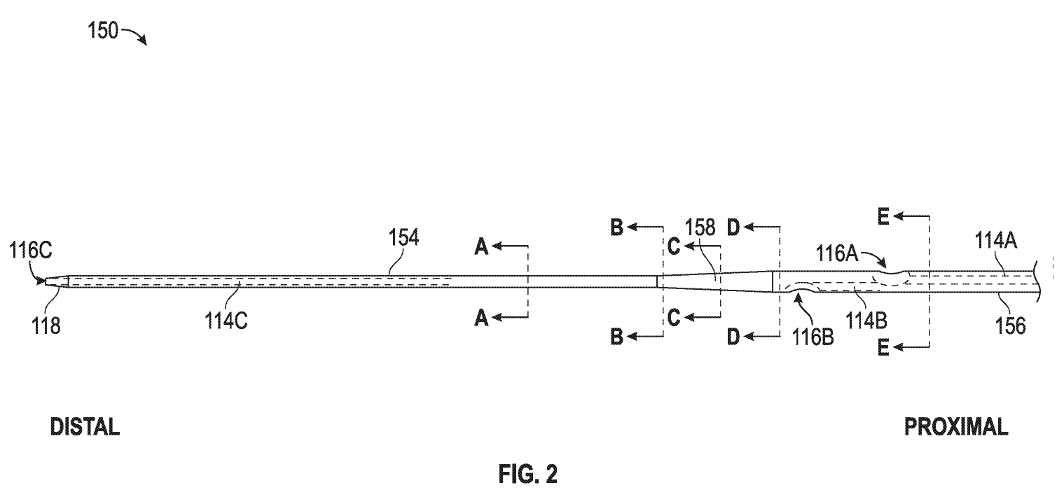

FIG. 2 shows further details of a distal portion of the RICC catheter 150, including the access section 154, the dilator section 158, and a distal portion of the multi-luminal section 156. In an embodiment, the multi-luminal section 156 can include a proximal lumen 114A terminating at a proximal lumen aperture 116A, and a medial lumen 114B terminating at a medial lumen aperture 116B. Each of the proximal lumen aperture 116A and the medial lumen aperture 116B can extend through a side wall of the multi-luminal section 156. Each of the proximal lumen aperture 116A and the medial lumen aperture 116B can be disposed proximally of the dilator section 158. In an embodiment, the proximal lumen aperture 116A can be disposed proximally of the medial lumen aperture 116B. In an embodiment, the proximal lumen aperture 116A and the medial lumen aperture 116B can be disposed equidistant from the catheter hub 160.

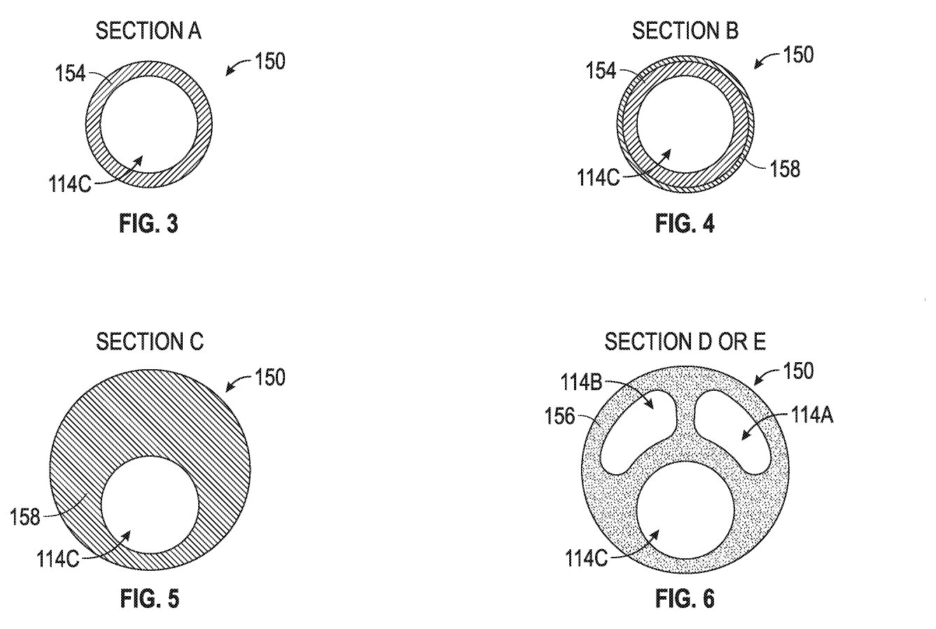

FIG. 3 shows a cross section view of the access section 154 at point “A” of FIG. 2. As shown, the access section 154 can define a single lumen and a relatively smaller outer diameter. A distal lumen 114C of the RICC catheter 150 can extend to a distal tip 118 of the RICC catheter 150 and can communicate with a distal lumen aperture 116C.

FIG. 4 shows a cross section view of the junction between the access section 154 and the dilator section 158, at point “B” of FIG. 2, where a portion of the access section 154 is received within the dilator section 158.

FIG. 5 shows a cross section view of the dilator section 158 at point “C” of FIG. 2, where an axis of the distal lumen 114C is offset from an axis of the dilator section 158 as the distal lumen 114 transitions between the multi-luminal section 156 and the access section 154.

FIG. 6 shows a cross section view of the multi-luminal section 156 at points “D” or “E” of FIG. 2, showing the proximal lumen 114A, the medial lumen 114B and the distal lumen 114C.

### Tip forming methods

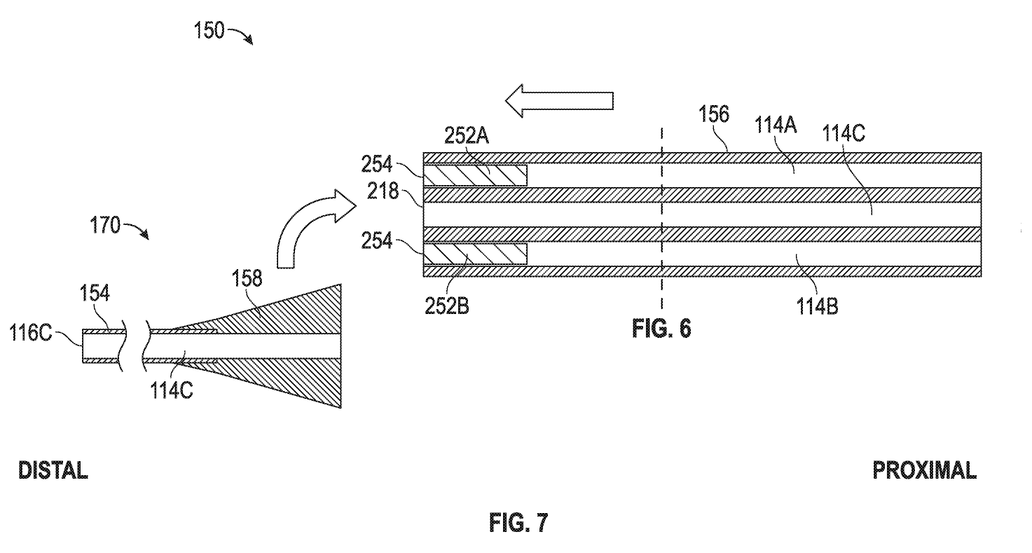

FIG. 7 shows an exemplary method of manufacturing a catheter 150 including a distal tip structure 170 having one or both of the access section 154 and the dilator section 158. In an exemplary method of coupling a distal tip structure 170 to a multi-luminal section 156 to form a catheter body 152, termed “tipping,” the multi-luminal section 156 can be formed having one or more lumen 114. In an embodiment, the multi-luminal section 156 can be extruded and trimmed to a desired length. It will be appreciated, however, that other methods of forming a multi-luminal section 156 are also contemplated. As shown in FIG. 7, a triple lumen multi-luminal section 156 is provided including a first (proximal) lumen 114A, a second (medial) lumen 114B, and a third (distal) lumen 114C. However, it will be appreciated that other single or multi-lumen catheters 150 are also contemplated. To note, the lumen 114A, 114B, 114C of the multi-luminal section 156 can be arranged radially about a central axis of the multi-luminal section 156, as shown in FIG. 6. In FIG. 7, the lumens 114A, 114B, 114C are shown adjacent to each other for clarity. It will be appreciated, however, that other configurations of multi-lumen catheters 150 are also contemplated.

In an embodiment, a plug 252 can be disposed into one or more lumen 114. For example, a first plug 252A can be disposed within a distal end of a first lumen 114A, and a second plug 252B can be disposed within a distal end of a second lumen 114B. A distal tip 254 of the plug 252 can align with a distal end 218 of the multi-luminal section 156. Optionally, a distal tip 254 of the plug 252 can be trimmed to align with a distal end 218 of the multi-luminal section 156.

The distal tip structure 170 can then be coupled with a distal end 218 of the multi-luminal section 156 using adhesive, bonding, solvent bonding, welding or the like. A lumen of the distal tip structure 170 can align with a lumen of the multi-luminal section 156 to form a distal lumen 114C extending to a distal lumen aperture 116C. The first plug 252A can seal a distal end of the proximal lumen 114A, and the second plug 252B can seal a distal end of the medial lumen 114B, proximally of the dilator section 158. The proximal lumen aperture 116A can then be formed through a wall of the multi-luminal section 156 and communicate with the proximal lumen 114A. The medial lumen aperture 116B can then be formed through a wall of the multi-luminal section 156 and communicate with the medial lumen 116B.

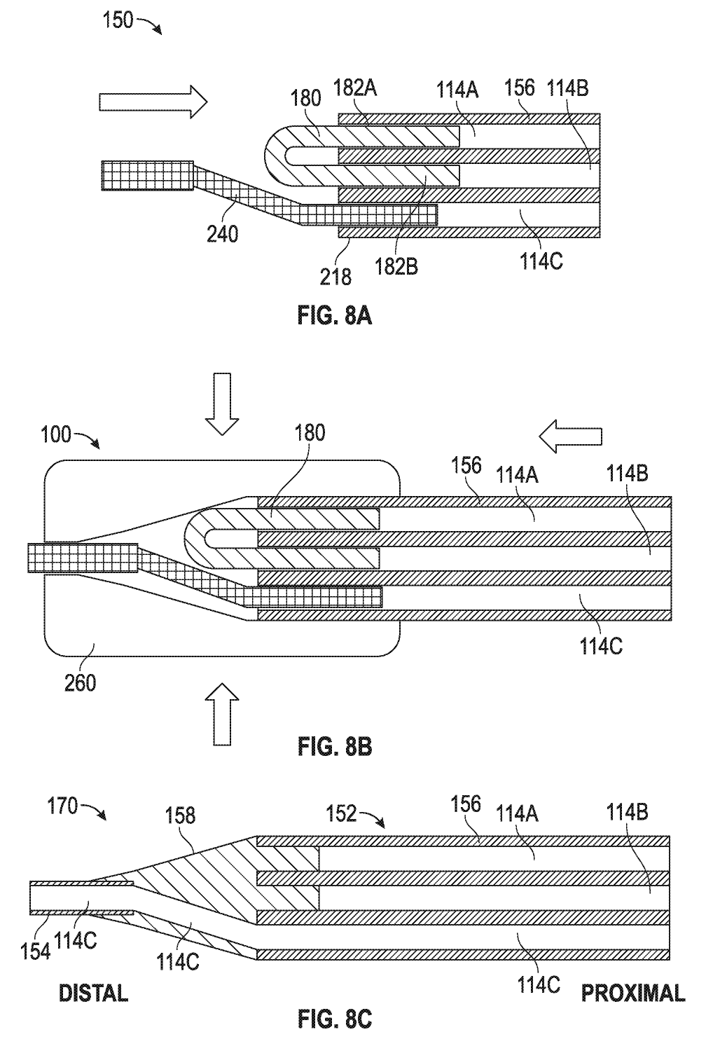

FIGS. 8A-8C show an exemplary method of forming a catheter 150 having a distal tip structure 170 including one or more of a dilator section 158 and an access section 154. As shown in FIG. 8A, a multi-luminal section 156 of the catheter body 152 is formed including one or more lumen 114, as described herein. In an embodiment, the multi-luminal section 156 can be formed of a first material.

In an embodiment, a bifurcated plug 180 can be provided including a second material different from the first material, and including one or more tines 182. As shown in FIG. 8A, the bifurcated plug 180 can include a first tine 182A and a second tine 182B. The first tine 182A can be disposed within a first lumen 114A, and the second tine 182B can be disposed within a second lumen 114B. In an embodiment, the bifurcated plug 180 can include a polymeric rod or a polymeric tube folded in half with a first end defining the first tine 182A and a second end defining the second tine 182B. To note, a distal end of the bifurcated plug 180 can extend distally of a distal end 218 of the multi-luminal section 156. In an embodiment, a proximal end of a mandrel 240 can be placed in the third lumen 114C and can define a pathway for a portion of the distal lumen 114C as it extends from the multi-luminal section 156 to the access section 154.

As shown in FIG. 8B, the distal end 218 of the multi-luminal section 156, including one or both of the bifurcated plug 180 and the mandrel 240, can then be placed within a die 260. Energy (thermal energy, radio frequency (RF) energy, ultrasonic, or the like) and/or pressure can be applied to the distal end 218 of the multi-luminal section 156 and the bifurcated plug 180 assembly to plastically deform the bifurcated plug 180 into at least a portion of the distal tip structure 170. The mandrel 240 can define a portion of the distal lumen 114C extending therethrough. In an embodiment, the distal tip structure 170 can define one or both of the dilator section 158 and the access section 154. In an embodiment, the distal tip structure 170 can include the dilator section 158 and can define a recess configured to receive a proximal end of the access section 154. Optionally, the access section 154 can be formed of a third material, different from both the first material and the second material and displaying different mechanical properties, durometers, or the like. The proximal end of the access section 154 can then be coupled with the dilator section 158 using adhesive, bonding, welding, or the like.

Advantageously, the material of the bifurcated plug 180 used to occlude the proximal lumen 114A and the medial lumen 114B, can be used to form the dilator section 158. As such, only a single structure is required to both occlude the lumen and form the distal tip structure 170, rather than two or more separate structures. This simplifies the manufacturing process and reduces associated costs.

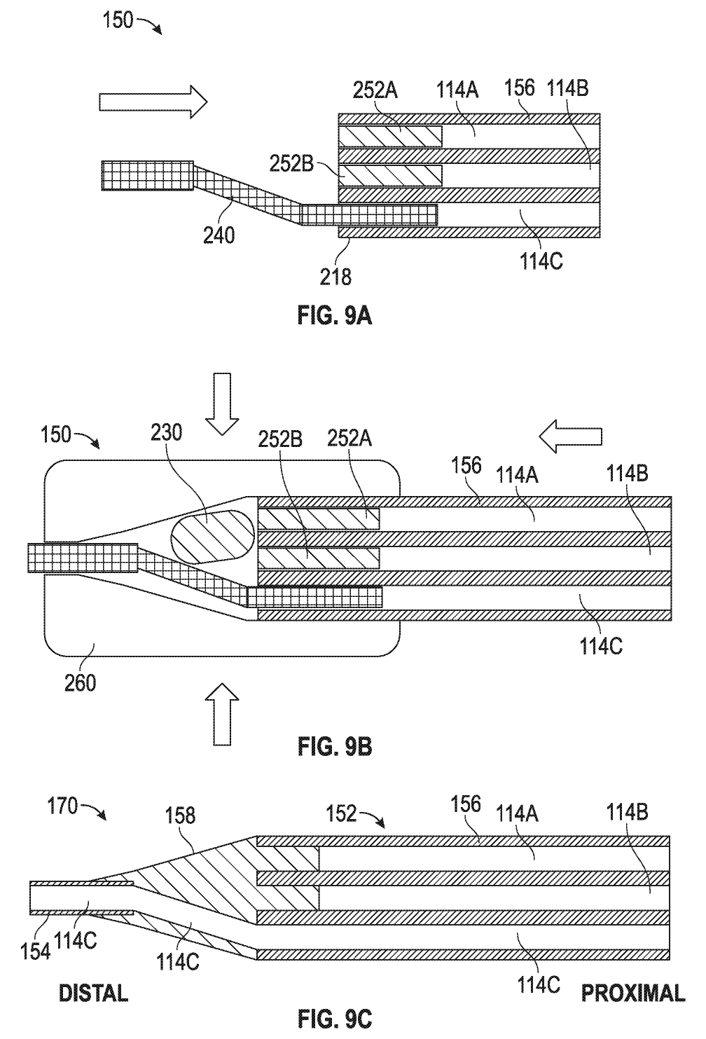

In an embodiment, as shown in FIGS. 9A-9C, the multi-luminal section 156 and mandrel 240 assembly can be provided, as described herein. The multi-luminal section 156 can be formed of a first material. The multi-luminal section 156 can include one or more plugs 252. The multi-luminal section 156, plug(s) 252, and mandrel 240 assembly can then be placed within the die 260 along with a pellet 230 of polymeric material formed of a second material within the die 260. Energy (thermal, RF, ultrasonic, or the like) and/or pressure can be applied to the distal end 218, mandrel 240, and pellet 230 assembly to plastically deform the pellet 230 into the distal tip structure 170.

In an embodiment, the multi-luminal section 156 can be formed of a first material and the plug 252 and/or pellet 230 can be formed of a second material, different from the first material. In an embodiment, the second material can display different mechanical properties from the first material. In an embodiment, one of the first material, second material, or third material can be a plastic, polymer, polyurethane, composite, elastomer, or the like. In an embodiment, the second material can display more rigid, or harder durometer, mechanical properties relative to the first material that can display more compliant or softer durometer mechanical properties. In an embodiment, the third material can display more or less rigid, or greater or lesser durometer, relative to one or both of the first material and the second material.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method of forming a catheter, comprising:
extruding a catheter body from a first material, the catheter body including a first lumen, a second lumen, and a third lumen;
forming a distal tip onto the catheter body, comprising:
positioning a proximal end of a mandrel in a distal end of the third lumen;
positioning a portion of the mandrel, a distal end of the catheter body, and a pellet of a second material in a die, wherein the second material is different from the first material;
deforming the pellet around the mandrel to form the distal tip; and
removing the mandrel from the catheter body and the distal tip, wherein the third lumen extends from the distal end of the catheter body through the distal tip.

2. The method according to claim 1, wherein the first material is more compliant, or of a softer durometer, relative to the second material.

3. The method according to claim 1, wherein deforming the pellet around the mandrel to form the distal tip forms a dilator section that tapers distally from a first diameter at the distal end of the catheter body to a smaller second diameter.

4. The method according to claim 3, wherein deforming the pellet around the mandrel to form the distal tip forms a recess at a distal end of the dilator section, wherein the recess is in fluid communication with the third lumen.

5. The method according to claim 4, further comprising inserting an access section into the recess such that the access section is in fluid communication with the third lumen.

6. The method according to claim 5, wherein inserting the access section into the recess comprises positioning a distal end of the access section distal of the distal end of the dilator section.

7. The method according to claim 5, wherein the access section is formed of a third material, different from the first material and the second material.

8. The method according to claim 5, further comprising coupling the access section with the dilator section via adhesive, bonding, welding, or the like.

9. The method according to claim 1, wherein deforming the pellet around the mandrel to form the distal tip comprises applying one or more of pressure, thermal energy, radio frequency energy, or ultrasonic energy to plastically deform the pellet.

10. The method according to claim 1, wherein forming the distal tip onto the catheter body further comprises occluding the first lumen and the second lumen at the distal end of the catheter body by inserting a first plug into a distal end of the first lumen and inserting a second plug into a distal end of the second lumen.

11. A method of forming a distal tip for a catheter, comprising:
providing a catheter body formed from a first material, the catheter body including a first lumen and a second lumen;
forming a distal tip onto the catheter body, comprising:
positioning a proximal end of a mandrel in a distal end of the first lumen;
positioning a portion of the mandrel, a distal end of the catheter body, and a pellet of a second material in a die, wherein the second material is different from the first material;
deforming the pellet around the mandrel to form the distal tip; and
removing the mandrel from the catheter body and the distal tip, wherein the first lumen extends from the distal end of the catheter body through the distal tip.

12. The method according to claim 11, wherein the first material is more compliant, or of a softer durometer, relative to the second material.

13. The method according to claim 11, wherein deforming the pellet around the mandrel to form the distal tip forms a dilator section that tapers distally from a first diameter at the distal end of the catheter body to a smaller second diameter.

14. The method according to claim 13, wherein deforming the pellet around the mandrel to form the distal tip forms a recess at a distal end of the dilator section, wherein the recess is in fluid communication with the third lumen.

15. The method according to claim 14, further comprising inserting an access section into the recess such that the access section is in fluid communication with the third lumen.

16. The method according to claim 15, wherein inserting the access section into the recess comprises positioning a distal end of the access section distal of the distal end of the dilator section.

17. The method according to claim 15, wherein the access section is formed of a third material, different from the first material and the second material.

18. The method according to claim 15, further comprising coupling the access section with the dilator section via adhesive, bonding, welding, or the like.

19. The method according to claim 1, wherein deforming the pellet around the mandrel to form the distal tip comprises applying one or more of pressure, thermal energy, radio frequency energy, or ultrasonic energy to plastically deform the pellet.

20. The method according to claim 1, wherein forming the distal tip onto the catheter body further comprises occluding the second lumen by inserting a plug into a distal end of the second lumen.
